(19) United States
(12) Patent Application Publication
(10) Pub. No.: US 2023/0313250 A1
(43) Pub. Date: Oct. 5, 2023

(54) GALACTO-OLIGOSACCHARIDE HAVING A TERMINAL MANNOSE RESIDUE, ITS PREPARATION AND APPLICATION

(71) Applicant: FrieslandCampina Nederland B.V., Amersfoort (NL)

(72) Inventors: Linqiu CAO, Wageningen (NL); Johannes Adrianus Henricus Petrus BASTIAANS, Wageningen (NL)

(21) Appl. No.: 17/801,895

(22) PCT Filed: Mar. 3, 2021
(86) PCT No.: PCT/EP2021/055234

§ 371 Date: Aug. 24, 2022

(30) Foreign Application Priority Data
Mar. 4, 2020 (EP) 20160860.1

(51) Int. Cl. C12P 19/04; C07H 3/06; C07H 3/04; C07H 1/00; C12P 19/12; C12N 9/90; C12N 9/38; C08B 37/00; A23L 33/125

(52) U.S. Cl. CPC C12P 19/04; C07H 3/06; C07H 3/04; C07H 1/00; C12P 19/12; C12N 9/90; C12Y 501/03011; C12N 9/2471; C12Y 302/01023; C08B 37/0087; A23L 33/125

(57) ABSTRACT

The present invention provides a new type of galacto-oligosaccharide, having a mannose residue instead of a glucose residue at the reducing end. The invention also relates to compositions comprising this galacto-oligosaccharide, its preparation and use in nutritional compositions.

The present invention relates to a new galacto-oligosaccharide, its preparation, and its use in a nutritional composition.

GOS, also known as oligogalactosyllactose, oligogalactose, oligolactose or transgalactooligosaccharides (TOS), is an important food ingredient. Because of its indigestible nature, GOS belongs to the group of prebiotics. Prebiotics are defined as non-digestible food ingredients that beneficially affect the host by stimulating the growth and/or activity of beneficial bacteria in the colon. The ability of GOS, when added to infant milk formulas, to replicate the bifidogenic effect of human milk—not only in bacterial numbers, but also with respect to the metabolic activity of the colonic microbiota—has significantly increased interest in its production and application in various food and pharmaceutical processes. For example, GOS occurs in commercially available food products for both infants and adults, ranging from infant formula to food for the critically ill.

Conventional GOS comprises a chain of galactose units and a terminal glucose unit. Hence, it has the general formula (Gal)ₙGlu. In the individual chains, n has a value in the range 1-8; the average value of n in conventional GOS is higher than 1, meaning that it contains oligosaccharides of the formula (Gal)ₙGlu wherein n=2-8 and, generally, disaccharides of the formula Gal-Glu.

GOS synthesis typically involves a number of galactosyl transfer processes catalyzed by ß-galactosidase (ß-D-galactohydrolase; EC 3.2.1.23), which uses lactose as galactosyl donor and lactose or the intermediate GOS species as galactosyl acceptor. Examples of such beta-galactosidase enzymes are Bacillus circulans, Aspergillus oryzae, Aspergillus niger, Kluyveromyces marxianus, Kluyveromyces fragilis, Sporobolomyces singularis, Lactobacillus fermentum, and Papiliotrema terrestris.

Various physiological functions of GOS have been reported, including the capacity to stimulate the growth of bifidogenic bacteria in the gut, to support normal gut transit, to contribute to natural defenses and to enhance mineral absorption. GOS has received particular attention for its prebiotic effects that promote the growth of Bifidobacterium, Lactobacillus, and other enteric bacteria. Therefore, GOS is commonly used in infant formula, beverages fermented by Lactobacillus, and yoghurts. Some of these GOS-containing foods are certified as Food for Specified Health Uses by the Consumer Affairs Agency in Japan, and GOS is certified as generally recognized as safe (GRAS) substances by the U.S. Food and Drug Administration (GRAS Notices: GRN 233, 236, 285, 286, 334, 484, 489, 495, 518, and 569).

The present invention provides a new type of galacto-oligosaccharide, having a mannose residue instead of a glucose residue at the reducing end.

Mannose is a non-digesting sugar that is known to act as natural sugar antibiotic. For instance, it is widely used to treat urine tract infections. Bioactive proteins such as lactoferrin often contain glycans with mannose groups. Adhesins of pathogens such as Salmonella can recognize mannose groups and the human innate immune system is geared to recognize the mannose immunity.

Therefore, the new type of galacto-oligosaccharide according to the invention is expected to have an immunomodulating and pathogen inhibiting effect comparable with human colostrum.

The present invention therefore relates to a galacto-oligosaccharide of the formula (Gal)ₘ-Man, wherein m has a value in the range 2-8, preferably 2-5, provided that the galacto-oligosaccharide is not epilactose (Gal(ß1-4)Man).

The galacto-olgosaccharide according to this invention is abbreviated as M-GOS.

The galactose residues can form linear or branched chains. The mannose reducing end can be positioned at the end of the chain, or within the chain.

The galacto-oligosaccharide according to the present invention differs from the galacto-oligosaccharide disclosed in EP1887017 in that it contains a mannose unit at the reducing end, while the backbone is solely made of galactose units. The galacto-oligosaccharide of EP1887017, on the other hand, has the formula Galₙ-Xₒ-Galₚ (X can be mannose), and is prepared by reacting lactose with X (e.g. mannose) in the presence of ß-galactosidase. This means that many mannose units can be present in this prior art oligosaccharide, also in the backbone, and that the mannose is not necessarily positioned at the reducing end.

The galacto-oligosaccharide according to the present invention is preferably obtained as a mixture comprising (Gal)ₘ-Man with different values of m and/or different degrees of branching. Therefore, the invention also relates to a composition (i.e. an M-GOS-containing composition) comprising:
(i) at least one galacto-oligosaccharide of the formula (Gal)ₘ-Man wherein m has a value in the range 2-8, preferably 2-5, and
(ii) at least one galacto-disaccharide of the formula Gal-Man, preferably selected from Gal(ß1-4)Man (i.e. epilactose), Gal(ß1-2)Man, Gal(ß1-3)Man, and Gal(ß1-6)Man.

This composition preferably comprises 0.1-99 wt %, more preferably 5-95 wt %, and most preferably 10-50 wt % galacto-oligosaccharides of the formula (Gal)ₘ-Man wherein m has a value in the range 2-8, preferably 2-5, and preferably 1-99.9 wt %, more preferably 5-95 wt %, and most preferably 50-90 wt % galacto-disaccharides of the formula Gal-Man; all based on the total weight of oligosaccharides and disaccharides in the composition.

In a preferred embodiment, the invention relates to an M-GOS-containing composition comprising (i) M-GOS or the M-GOS containing composition as defined above, in admixture with (ii) one or more galacto-oligosaccharides of the formula (Gal)ₙ-Glu (GOS), wherein n has a value in the range 1-8. Preferably, this composition comprises one or more galacto-oligosaccharides of the formula (Gal)ₙ-Glu, wherein n has a value in the range 2-8, and one or more disaccharides of the formula Gal-Glu, such as lactose.

More preferably, the total content of compounds with the formula (Gal)ₘ-Man with m being in the range 1-8 in said composition is at least 3 wt %, preferably at least 5 wt %, even more preferably at least 10 wt %, and most preferably 10-50 wt %, based on the total weight of oligosaccharides and disaccharides in the composition.

The total weight of oligosaccharides and disaccharides is the total weight of all carbohydrates built up from two or more monosugar residues, and thus includes GOS, M-GOS, Gal-Glu disaccharides such as lactose and Gal-Man disaccharides such as epilactose.

Said M-GOS and M-GOS-containing compositions can be prepared by three different methods.

According to the first method, a lactose-containing feed is contacted with an epimerase enzyme. As a result, the lactose in the feed is converted into epilactose. The formed epilactose is purified and then converted into M-GOS using a beta-galactosidase enzyme.

According to the second method, a lactose-containing feed is contacted with both an epimerase enzyme and a beta-galactosidase enzyme. This method thus differs from the first method in that the epilactose that is formed is not purified before being contacted with the beta-galactosidase enzyme. Both reaction steps can thus be performed simultaneously, in the same reactor (one-pot synthesis).

According to the third method, a feed comprising GOS with the formula (Gal)ₙGlu wherein n=2-8, optionally in admixture with a galacto-disaccharide with the formula Gal-Glu, is contacted with an epimerase enzyme to form a mixture comprising epilactose, regular GOS, and M-GOS. This method thus allows to add a new functionality to existing GOS.

These methods differ from the method disclosed in EP1887017 discussed above in that no mannose needs to added; mannose is a quite expensive raw material. Instead, the processes according to the present invention allow for the use of lactose or GOS as the sole raw material.

Lactose-Containing Feed

The lactose-containing feed used in the first and second method can be an aqueous lactose solution or an aqueous suspension of lactose crystals. The lactose concentration in said solution or suspension is preferably 15-75 wt %, more preferably 40-75 wt %, more preferably 50-70 wt % wt %, and most preferably 55-70 wt %. The pH of the slurry or solution is preferably in the range 3.5-7.5, more preferably 5.0-7.5.

The lactose can be food grade, pharmaceutical grade, or refined. Food grade lactose is conventionally produced by concentrating whey or whey permeate (a co-product of whey protein concentrate production) to form a supersaturated solution from which lactose crystalizes out. The lactose crystals are then removed and dried.

Refined or a pharmaceutical grade lactose can be obtained by re-dissolving the lactose crystals and treating the solution with virgin activated carbon, which absorbs a number of solutes (including riboflavin and a variety of proteins) and proteose peptones (polypeptides are derived from ß-casein), followed by further crystallization and washing steps.

Alternatively, the lactose-containing feed can be a lactose-containing whey permeate, such as cheese whey permeate (CWP) or a CWP that has been processed further to remove unwanted components and/or to enrich for desirable components. CWP is a lactose-rich effluent remaining after protein extraction from cheese whey, an abundant dairy waste. In all milk-producing countries, milk is primarily used to manufacture cheese. However, only approximately half of the solids present in milk are coagulated and recovered as cheese; the remaining half are recovered as whey. Whey contains mainly proteins, lactose, minerals and vitamins Upon ultrafiltration (UF) of the whey, commercially valuable proteins are collected from the UF-retentate; the UF-permeate is the cheese whey permeate; also known as liquid permeate. Cheese whey permeate contains mainly lactose, minerals and vitamins.

CWP can be used as the lactose-containing feed as such, i.e. without further treatment, or after demineralization. In one embodiment, the lactose-containing feed is a CWP that is demineralized to an ash content of up to about 4 wt. %, or a conductivity of up to about 4 mS. Demineralization may be performed by methods known in the art, including electrodialysis (ED), reverse osmosis (RO), nanofiltration (NF) or ion exchange technology.

The (demineralized) CWP that may be used as the lactose-containing feed has preferably been concentrated to a dry matter content of at least 25 wt %, preferably at least 50 wt % or more.

In a further alternative embodiment, the lactose-containing feed may be delactosed whey permeate (DLP or OPL), which still contains lactose and protein, but also the minerals and vitamins originally present in the whey permeate. OPL also contains 0.3-0.4% sialyl-lactose (2,3-sialylactose and 2,6-sialyl lactose) and possibly other valuable bovine milk oligosaccharides (bMOs) as well. Besides, it has been shown that bMOs contain as many oligosaccharides as found in human oligosaccharides (hMOs).

OPL contains relatively low amounts of calcium. Therefore, in order to remove multivalent anions such as P043-, citrate 3-, extra calcium can be added to enhance the formation of insoluble calcium monohydrogen phosphate and calcium citrate, thus allowing for easy removal of salts by e.g. centrifugation. In a preferred embodiment, OPL is treated with lime (CaO/Ca(OH)₂) to precipitate anions.

The Epimerase Enzyme

The epimerase enzyme to be used in the first, second, and third method can be any epimerase enzyme that is able to convert the glucose-reducing end of lactose or galacto-oligosaccharides into a mannose reducing end. An example of a suitable epimerase enzyme is cellobiose 2-epimerase.

The enzyme can be used as such, or in immobilized form. Various ways of enzyme immobilization are known in the art. They typically comprise a porous carrier onto which the beta-galactosidase is immobilized via covalent binding, via physical absorption (charge-charge or van der Wags interaction), via gel encapsulation or a combination thereof. Examples of suitable solid carriers are activated acrylic polymers, preferably functionalized polymethacrylate matrices such as hexamethylenamino-functionalized polymethacrylate matrices (Sep abeads) or macroporous acrylic epoxy-activated resins like Eupergit C 250L.

Besides, carrier-free immobilized enzymes such as CLEC (cross-linked enzyme crystals) or CLEAs (cross-linked enzyme aggregates) might be also applied.

The Beta-Galactosidase Enzyme

The beta-galactosidase enzyme that can be used in all methods referred to above can be any suitable beta-galactosidase enzyme, such as those isolated from a micro-organism selected from the group consisting of Bacillus circulans, Aspergillus oryzae, Aspergillus niger, Kluyveromyces marxianus, Kluyveromyces fragilis, Sporobolomyces singularis, Lactobacillus fermentum, Bifidobacterium breve, Bifidobacterium bifidum, Bifidobacterium infantis and Papiliotrema terrestris.

Some of the enzymes have high specificity to synthesize oligosaccharides of specific chain length and orientation of the linkage. For example, ß-galactosidase sourced from B. circulans shows high specificity to ß-1,4 linkages and in turn yields mainly ß-1,4 linked galactosyl oligosaccharides by transglycosylation, while ß-galactosidase sourced from Aspergillus oryzae gives ß-1,6 linkages mainly.

The enzyme can be used as such, or in immobilized form. Various ways of enzyme immobilization are known in the art. They typically comprise a porous carrier onto which the beta-galactosidase is immobilized via covalent binding, via physical absorption (charge-charge or van der Wags interaction), via gel encapsulation or a combination thereof. Examples of suitable solid carriers are activated acrylic polymers, preferably functionalized polymethacrylate matrices such as hexamethylenamino-functionalized polymethacrylate matrices (Sep abeads) or macroporous acrylic epoxy-activated resins like Eupergit C 250L.

Besides, carrier-free immobilized enzymes such as CLEC (cross-linked enzyme crystals) or CLEAs (cross-linked enzyme aggregates) might be also applied.

Method 1

The conversion of lactose into epilactose in the first method can be suitably performed at a temperature in the range 40-70° C.

The amount of enzyme is preferably in the range 0.1-50 U/gram substrate, more preferably 0.5-20 U/gram substrate, and most preferably 1-10 U/gram substrate.

The reaction time is preferably 5-40 hours, more preferably 8-30 hours, and most preferably 10-20 hours, depending on substrate concentration, enzyme dosage, and reaction temperature.

The purification of the epilactose in the first step of the first method can be performed by crystallization of the lactose.

The reaction with beta-galactosidase enzyme in the second step of the first method can be suitably performed at a temperature in the range 20-75° C., preferably 30-70° C., and most preferably 40-60° C.

The enzyme dosage is preferably at least 0.60 LU/gram substrate, more preferably in the range 0.60-1.1 LU/gram, even more preferably 0.65-1.0 LU/gram, and most preferably 0.65-0.75 LU/gram. As used herein, one lactase unit (LU) is defined as the quantity of enzyme that liberates 1 μmole of glucose per minute at the early stage of the reaction at 40° C., pH 6.0. When lactose is hydrolysed by lactase, it is converted into glucose and galactose. The lactase activity is determined by measuring the amount of liberated glucose.

The reaction time is preferably at least 10 hours, more preferably 10-30 hours, and most preferably 20-26 hours, depending on substrate concentration, enzyme dosage, and reaction temperature.

The substrate concentration, i.e. the total carbohydrate concentration, is preferably in the range 4-70 wt %, more preferably 20-40 wt %, and most preferably in the range 50-60? wt %. If the substrate concentration is lower, hydrolysis of epilactose will dominate over M-GOS formation

Method 2

The one-pot synthesis of the second method, during which both epimerase enzyme and beta-galactosidase enzyme are present, is preferably performed at a temperature in the range 40-70° C., preferably 50-65° C., and most preferably 5-60° C. The amount of epimerase enzyme is preferably in the range 0.1-10 U/gram lactose, more preferably 0.5-5 U/gram lactose, and most preferably 1-2.5 U/gram lactose. The amount of beta-galactosidase enzyme is preferably in the range 0.5-10 U/gram lactose, more preferably 0.75-7.5 U/gram lactose, and most preferably 1.0-3.5 U/gram lactose.

The reaction time is preferably 10-50 hours, more preferably 15-42 hours, and most preferably 20-24 hours, depending on substrate concentration, enzyme dosage, and reaction temperature.

The substrate concentration, i.e. the total carbohydrate concentration, is preferably in the range 4-70 wt %, more preferably 20-40 wt %, and most preferably in the range 50-60 wt %. If the substrate concentration is lower, hydrolysis of epilactose will dominate over M-GOS formation.

Method 3

The GOS-containing feed that is used in the third method is preferably an aqueous feed comprising at least 30 wt %, more preferably 50-60 wt % GOS, i.e. compounds with the formula (Gal)ₙ-Glu with n being in the range 2-8, or—preferably—a composition comprising such compounds in admixture with disaccharides of the formula Gal-Glu (e.g. lactose). In addition, the feed preferably also contains monosugars, including glucose and galactose.

The third method is preferably performed at a temperature in the range 40-70° C., preferably 50-65° C., and most preferably 55-60° C.

The amount of epimerase enzyme in the third method is preferably in the range 0.1-50 Unit/gram GOS, more preferably 0.5-20 Unit/gram GOS, and most preferably 1-10 Unit/gram GOS. The reaction time is preferably 10-30 hours, more preferably 10-30 hours, and most preferably 20-26 hours,

The M-GOS-containing compositions that are formed by the methods according to the present invention can be purified from the reaction mixture by denaturing the enzyme(s)—for instance by heat treatment (e.g. 100° C. for 15 minutes) or acidification—demineralization, de-proteinization, removal of mono-sugar components, and/or decolouration (e.g. by treatment with activated carbon). In one embodiment, the M-GOS-containing composition is subjected to a nanofiltration (NF) or ultrafiltration step (UF) to remove mono-sugars and protein. A further concentration step, for example using NF or evaporation, may be performed to obtain a concentrated M-GOS preparation having a dry matter content of, e.g., at least 70 wt %, preferably at least 75 wt %.

Alternatively, only enzyme residues are removed from the reaction mixture, but no further purification or isolation of individual components is applied.

M-GOS and M-GOS-containing compositions can be used in nutritional compositions. This can be nutritional compositions for pregnant women (MUM compositions), young children (e.g. infant formula, follow-up formula, or growing up milk (GUM)), adolescents (13-20 years of age), or adults (>20 years of age). The nutritional composition can be used as a regular food composition, as nutritional therapy, as nutritional support, as medical food, as a food for special medical purposes, or as a nutritional supplement.

The nutritional compositions may further comprise proteins (dairy proteins and/or plant proteins, either hydrolyzed or unhydrolysed), probiotics, lipid sources, and/or further carbohydrates, such as lactose, saccharose, starch or maltodextrin.

Examples of suitable lipid sources are tri, di, and monoglycerides, phospholipids, sphingolipids, fatty acids, and esters or salts thereof. The lipids may have an animal, vegetable, microbial or synthetic origin. Of particular interest are polyunsaturated fatty acids (PUFAs) such as gamma linolenic acid (GLA), dihomo gamma linolenic acid (DHGLA), arachidonic acid (AA), stearidonic acid (SA), eicosapentaenoic acid (EPA), docosahexaenoic acid (DHA), docosapentaenoic acid (DPA) and conjugated linoleic acid (CLA). CLA is important in the protection against eczema and respiratory diseases in children. This particularly involves the cis-9, trans-11 and cis-12 isomers of CLA. Examples of suitable vegetable lipid sources include sun flower oil, high oleic sun flower oil, coconut oil, palm oil, palm kernel oil, soy bean oil, etc. Examples of suitable lipid sources of animal origin include milkfat, for example anhydrous milkfat (AMF), cream, etc. In a preferred embodiment, a combination of milkfat and lipids of vegetable origin are used.

The nutritional composition may further comprise a probiotic. In the context of the present invention, the term “probiotic” refers to a strain of probiotic bacteria. Probiotic bacteria are known in the art. Suitably, the probiotic bacteria are not genetically modified. Suitable probiotic bacteria include bacteria of the genus Bifidobacteria (e.g. B. breve, B. longum, B. infantis, B. bifidum), Lactobacillus (e.g. L. Acidophilus, L. paracasei, L. johnsonii, L. plantarum, L. reuteri, L. rhamnosus, L. casei, L. lactis), and Streptococcus (e.g. S. thermophilus). B. breve and B. longum are especially suitable probiotics. Suitable B. breve strains may for example be isolated from the faeces of healthy human milk-fed infants.

The combination of a prebiotic and a probiotic is also referred to as a “synbiotic”. The probiotic may be present in the composition at any suitable concentration, suitably in a therapeutically effective amount or “amount effective for treating” in the context of the invention. Suitably, the probiotic is included in the nutritional composition in an amount of 10²-10¹³ cfu per g dry weight of the composition, suitably 10⁵-10¹² cfu/g, most suitably 10⁷-10¹⁰ cfu/g.

Further, the nutritional composition may contain one or more conventional micro ingredients, such as vitamins, antioxidants, minerals, free amino acids, nucleotides, taurine, carnitine and polyamines. Examples of suitable antioxidants are BHT, ascorbyl palmitate, vitamin E, alpha and beta carotene, lutein, zeaxanthin, lycopene and phospholipids.

### EXAMPLES

### Example 1

Aqueous lactose slurries of pH=6.5 were prepared by slurrying 81 g lactose crystals in a 65 g 1 M natrium citrate buffer solution. A beta-galactosidase (Bacillus circulans) and/or a cellobiose-2-epimerase were added in the amounts listed in Table 1. The slurries were stirred with a magnetic bar and thermostated at 59° C. in a water bath. After 20 hours, the reactions were stopped by adding 1.5% (v/v) 1 M HCl, in order to denature the enzyme.

The carbohydrate composition of the resulting mixtures was analyzed by Dionex HPLC and displayed in Table 2.

TABLE 1

| | Epimerase | Biolacta N5 |
|---|---|---|
| experiment | (U/gram lactose) | (LU/gram lactose) |
| A | | 3.90 |
| B | 1 | 3.90 |
| C | 1 | |
| D | 1 | 1.95 |

Experiment A resulted in the formation of a conventional galacto-oligosaccharide composition. Experiment C showed the formation epilactose. Experiments B and D resulted in additional peaks, not observed in experiments A and C, indicating the formation of M-GOS. Interestingly, it was found that with a reduced dosage of beta-galactosidase (experiment D), the formation of conventional GOS was suppressed, while increasing the percentage of M-GOS.

TABLE 2
Carbohydrate composition of the reaction mixture (in wt %)

| | Exp. A | Exp. B | Exp. C | Exp. D |
|---|---|---|---|---|
| gal | 1.8 | 1.42 | | 0.88 |
| gluc | 13.66 | 12.09 | | 12.76 |
| mannose | | 2.03 | | 1.54 |
| allolactose | 4.07 | 3.43 | | 2.19 |
| lactose | 20.7 | 14.57 | 49.23 | 19.97 |
| lactulose | 1.88 | 2.46 | 6.13 | 2.47 |
| epilactose | | 12.01 | 36.08 | 14.11 |
| GOS (m = 2-5) | 57.89 | 44.05 | | 40.37 |
| M-GOS (m = 2-5) | | 19.95 | | 22.29 |

### Example 2

Commercial Vivinal® GOS (ex-FrieslandCampina) was used to prepare 100 gram of an aqueous GOS solution, having a solids content of 54 wt % and a pH of 6.5. This pH was set by the addition of 1 ml 1M natrium citrate.

8 units of a cellobiose-2-epimerase (i.e. 1 Unit epimerase/gram lactose present in the said GOS solution) were added, to initialize the epimerization.

The slurries were stirred with a magnetic bar and thermostated at 59° C. in a water bath. After 20 hours, the reactions were stopped by adding 1.5% (v/v) 1 M HCl, in order to denature the enzyme.

The obtained product was analyzed by Dionex HPLC for its GOS profile and sugar composition by estimation of the peak percentage and the results are displayed in Table 3.

This experiment resulted in the formation of two extra components: epilactose and M-GOS. This shows that addition of epimerase to a commercial GOS product is able to convert the remaining lactose into epilactose and the lactose-based GOS into M-GOS. This means that addition of epimerase is not only able to introduce a novel functional component, but also to increase the total non-digestible oligosaccharide content.

TABLE 3
Carbohydrate composition of the reaction mixture (in wt %)

| Component | Vivinal ® GOS | Product of Example 2 |
|---|---|---|
| galactose | 2.01 | 2.27 |
| glucose | 13.49 | 12.66 |
| allo-lactose | 4.81 | 4.96 |
| lactose | 15.46 | 11.59 |
| lactulose | 1.44 | 2 |
| epilactose | | 6.06 |
| M-GOS (m = 2) | | 3.79 |
| GOS | 62.79 | 56.67 |

1. Galacto-oligosaccharide of the formula (Gal)ₘ-Man not being epilactose, wherein m has a value in the range 2-8.

2. Composition comprising (i) at least one galacto-oligosaccharide of the formula (Gal)m-Man, wherein m has a value in the range 2-8, and (ii) at least one galacto-disaccharide of the formula Gal-Man.

3. Composition according to claim 2 comprising 0.1-99 wt % galacto-oligosaccharides of the formula (Gal)m-Man, wherein m has a value in the range 2-8, and 1-99.9 wt % galacto-disaccharides of the formula Gal-Man based on the total weight of oligosaccharides and disaccharides in the composition.

4. Composition comprising (i) at least one galacto-oligosaccharide according to claim 1 in admixture with (ii) at least one galacto-oligosaccharide of the formula (Gal)n-Glu, wherein n has a value in the range 1-8.

5. Composition according to claim 4 comprising at least one galacto-oligosaccharide with the formula (Gal)n-Glu wherein n has a value in the range 2-8 and optionally at least one galacto-disaccharide with the formula Gal-Glu.

6. Composition according to claim 4 wherein the total content of compounds with the formula (Gal)m-Man with m being in the range 2-8 in said composition is at least 3 wt % based on the total weight of oligosaccharides and disaccharides.

7. Process for the preparation of the galacto-oligosaccharide according to claim 1, the process comprising the steps of:
(i) reacting a lactose with epimerase, thereby obtaining epilactose, and
(ii) reacting said epilactose with a β-galactosidase, thereby obtaining the galacto-oligosaccharide according to claim 1.

8. Process according to claim 7 wherein the epilactose formed in step (i) is purified before being contacted with the β-galactosidase in step (ii).

9. Process according to claim 8 wherein the steps (i) and (ii) are conducted in a one-pot reaction, thereby contacting lactose with both said epimerase and said β-galactosidase.

10. Process for the preparation of the galacto-oligosaccharide according to claim 1, the process comprising the step of contacting a galacto-oligosaccharide with the formula (Gal)n-Glu wherein n=2-8, optionally in admixture with a galacto-disaccharide with the formula Gal-Glu, with epimerase, thereby forming a galacto-oligosaccharide of formula (Gal)m-Man, wherein m=2-8 and optionally a galacto-disaccharide of the formula Gal-Man.

11. Nutritional composition comprising the galacto-oligosaccharide of claim 1, additionally comprising one or more proteins, probiotics, lipids, and/or further carbohydrates.

12. Galacto-oligosaccharide according to claim 1, wherein m has a value in the range 2-5.

13. Composition according to claim 2, wherein m has a value in the range 2-5.

14. Composition according to claim 3 comprising 5-95 wt % galacto-oligosaccharides of the formula (Gal)ₘ-Man, wherein m has a value in the range 2-5, and 5-95 wt % galacto-disaccharides of the formula Gal-Man based on the total weight of oligosaccharides and disaccharides in the composition.

15. Composition according to claim 4, wherein n has a value in the range 1-5.

16. Composition according to claim 6, wherein the total content of compounds with the formula (Gal)ₘ-Man with m being in the range 2-8 in said composition is at least 5 wt % based on the total weight of oligosaccharides and disaccharides.
